(19) United States
(12) Patent Application Publication
(10) Pub. No.: US 2026/0283686 A1
(43) Pub. Date: Sep. 24, 2026

(54) MULTI-FORM CATHETER

(71) Applicant: Biosense Webster (Israel) Ltd., Yokneam (IL)

(72) Inventors: Assaf GOVARI, Haifa (IL); Christopher Thomas BEECKLER, Brea, CA (US); Joseph Thomas KEYES, Sierra Madre, CA (US); Justin George LICHTER, Irvine, CA (US)

(73) Assignee: Biosense Webster (Israel) Ltd., Yokneam (IL)

(21) Appl. No.: 19/680,007

(22) Filed: May 18, 2026

Related U.S. Application Data
(63) Continuation of application No. 17/566,415, filed on Dec. 30, 2021, now Pat. No. 12,629,201.

(51) Int. Cl. A61B 18/14; A61B 17/00; A61B 18/00; A61M 25/00

(52) U.S. Cl. CPC A61B 18/1492; A61B 2017/00867; A61B 2018/00267; A61B 2018/00351; A61B 2018/00577; A61M 25/0074

(57) ABSTRACT

In one exemplary mode, a catheter apparatus includes an elongated deflectable element including a distal end, a flexible puller including a distal portion, and configured to be retracted through the deflectable element, and an expandable assembly including a plurality of resilient splines, each resilient spline including at least one electrode disposed thereon, the resilient splines being disposed circumferentially around the distal portion of the puller, with first ends of the splines being coupled with the distal end of the deflectable element and second ends of the splines coupled with the distal portion of the puller, the splines being configured to bow radially outward in a relaxed form of the expandable assembly and bow further radially outward when the puller is retracted expanding the expandable assembly from the relaxed form to an expanded form.

### CROSS-REFERENCE TO RELATED APPLICATIONS

This application claims priority under 35 U.S.C. § 120 to, and is a continuation of, U.S. Patent Application No. 17/566,415 filed Dec. 30, 2021 (Attorney Docket No. BIO6615USNP1_253757.238). The entire contents of which are hereby incorporated by reference.

### FIELD OF THE DISCLOSURE

The present disclosure relates to medical devices, and in particular, but not exclusively to, catheter devices.

### BACKGROUND

Cardiac arrhythmia, such as atrial fibrillation, occurs when regions of cardiac tissue abnormally conduct electric signals to adjacent tissue, thereby disrupting the normal cardiac cycle and causing asynchronous rhythm. Important sources of undesired signals are located in the tissue region along the pulmonary veins of the left atrium and in the superior pulmonary veins. In this condition, after unwanted signals are generated in the pulmonary veins or conducted through the pulmonary veins from other sources, they are conducted into the left atrium where they can initiate or continue arrhythmia.

Procedures for treating arrhythmia include surgically disrupting the origin of the signals causing the arrhythmia, as well as disrupting the conducting pathway for such signals. By mapping the electrical properties of the endocardium and the heart volume, and selectively ablating cardiac tissue by application of energy, it is sometimes possible to cease or modify the propagation of unwanted electrical signals from one portion of the heart to another. The ablation process destroys the unwanted electrical pathways by formation of non-conducting lesions.

In this two-step procedure, mapping followed by ablation, electrical activity at points in the heart is typically sensed and measured by advancing a catheter containing one or more electrical sensors into the heart, and acquiring data at a multiplicity of points. These data are then utilized to select the target areas at which ablation is to be performed commonly using the same catheter or a different catheter.

### BRIEF DESCRIPTION OF THE DRAWINGS

The present disclosure will be understood from the following detailed description, taken in conjunction with the drawings in which:

FIG. 1 is a schematic view of a catheter in a relaxed form constructed and operative in accordance with an exemplary mode of the present disclosure;

FIG. 2 is a schematic view of the catheter of FIG. 1 in an expanded form;

FIG. 3A is a more detail schematic view of the catheter of FIG. 1 in the relaxed form;

FIG. 3B is a plan view of the teardrop catheter of FIG. 1 with certain dimensions provided;

FIG. 3C is a plan view of the catheter of FIG. 1 with the distal end of the basket being pulled proximally to force the basket to assume a spheroidal shape;

FIG. 4 is a schematic view of the catheter of FIG. 1 being retracted into a sheath; and

FIG. 5 is a schematic cross-sectional view of the catheter of FIG. 1 in the sheath of FIG. 4.

### DESCRIPTION OF EXAMPLES

### OVERVIEW

Different catheter distal end shapes may perform different tasks better than others. For example, a more conical shape catheter distal end assembly may be more useful in conical regions of the heart, such as the pulmonary vein, whereas in other regions a more spherical shaped catheter distal end assembly may provide a larger diameter for a wider circumferential ablation. One solution is to insert different catheters into the heart chamber for different tasks. However, using different catheters may complicate the procedure and result in delays which could result in medical problems.

One solution is to provide a catheter, such as a basket catheter, which can change shape to accommodate the challenges presented by some of the different tasks. A common way to change the shape of the basket is to use a tube, e.g., polymer tube, which is fed through the catheter shaft and deflectable element and is connected to the distal end of the basket. The proximal end of the basket is connected to the distal end of the deflectable element. Advancing and retracting the tube through the shaft changes the shape of the basket. However, the tube makes the basket too rigid so that if the basket is pushed against the heart tissue, the basket does not deform sufficiently with respect to the tissue and electrodes of the catheter do not sufficiently contact the tissue for mapping and/or ablation purposes. Additionally, a rigid catheter could pose a trauma risk to the patient.

Therefore, some exemplary modes of the present disclosure solve at least some of the above problems by providing a catheter including an expandable assembly having resilient splines or spines that form a basket. The resilient splines bow outward when the expandable assembly is in a relaxed form. A flexible puller extends from a proximal end of the catheter to the distal end of the expandable assembly where the puller is coupled with the distal ends of the splines. The puller may be retracted (e.g., by about 0.5 centimeters depending on the size and configuration of the expandable assembly) to cause the splines to bow further radially outward expanding the expandable assembly from the relaxed form to an expanded form. As used herein, the term “splines” or “spines” means an elongated structural member with various cross-sections and such term “splines” or “spines” may be used interchangeably.

In some disclosed modes, the relaxed form of the expandable assembly defines a pear-shaped surface. In some disclosed modes, the pear-shaped surface comprises a convex-shaped region followed by a concave-shaped region in a direction progressing away from the distal end of the deflectable element. The pear shape is suited for narrower and/or conical regions of the heart chamber, e.g., for single shot pulmonary vein isolation. The expanded form has a more spherical shape more suited to wider circumferential ablations. Retracting the puller by different amounts may result in more than two forms of the expandable assembly.

As the catheter uses a flexible puller to adjust the shape of the expandable assembly, the assembly is very flexible and can be deformed when pressed against tissue so that there is sufficient contact between electrodes of the assembly and the tissue for mapping and/or ablation (such as irreversible electroporation IRE)).

The splines flatten and transform the expandable assembly from the relaxed form to a collapsed form when the expandable assembly is pulled into a sheath, which applies a radial compressive force on the splines. The puller is optionally stretchable and stretches as the splines are flattened in the collapsed form of the assembly. In some disclosed modes, the puller includes a stretchable woven tube. For example, if the catheter is about 1 meter long, and the length of the assembly increases by 1 centimeter between the relaxed form and the compressed form, the puller needs to stretch by about 1%.

Each resilient spline may include a spring alloy element, such as a nickel titanium elongated element, a spring tempered stainless steel element, or a beryllium copper (BeCu) element. The resilient spline optionally includes a polymer tube at least partially covering the spring alloy element. One or more electrodes may then be mounted on each spline on, or around, the polymer tube.

### SYSTEM DESCRIPTION

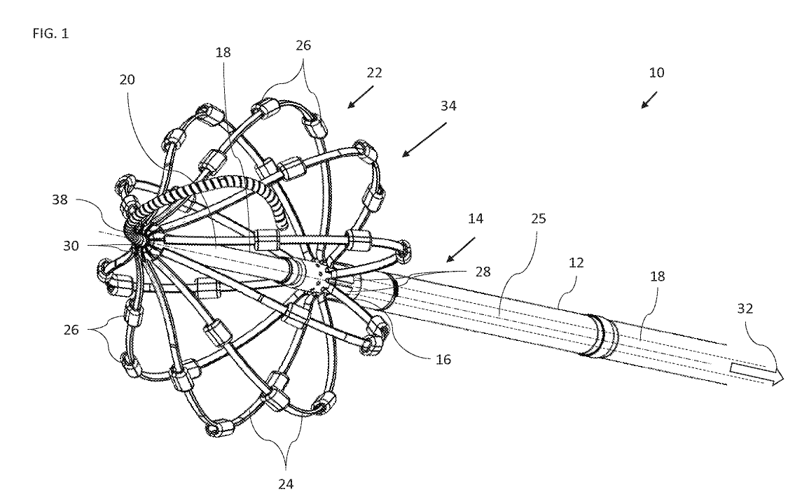

Reference is now made to FIG. 1, which is a schematic view of a catheter 10 in a relaxed form constructed and operative in accordance with an example of the present disclosure. The catheter 10 includes an elongated deflectable element 12 including a distal end 14. The catheter 10 may include a coupler 16 connected to the distal end 14 of the elongated deflectable element 12. The catheter 10 includes a flexible puller 18 including a distal portion 20, and configured to be retracted through the deflectable element 12. The catheter 10 may include a handle or manipulator (not shown) which is configured to retract the puller 18 in the elongated deflectable element 12 in the direction shown by arrow 32.

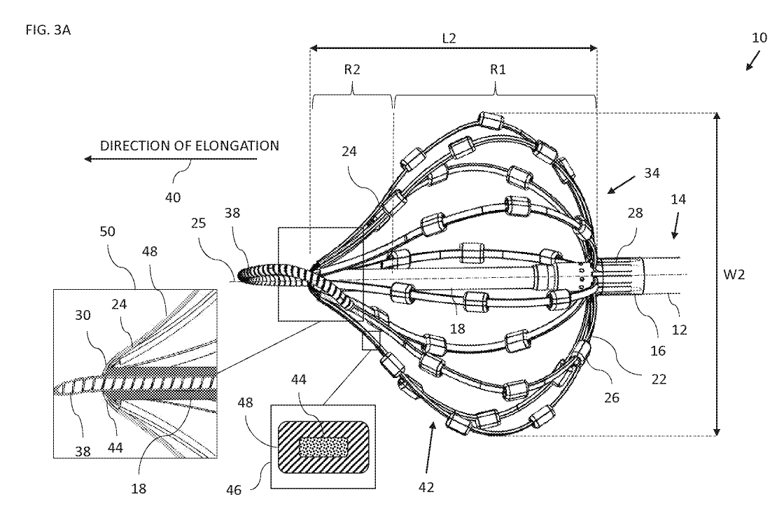

The catheter 10 includes an expandable assembly 22 comprising a plurality of resilient splines 24 (only two labeled for the sake of simplicity). Each resilient spline 24 includes at least one electrode 26 disposed thereon. In the example of FIG. 1, the expandable assembly 22 includes ten splines 24 with three electrode 26 disposed on each spline 24. The expandable assembly 22 may include any suitable number of splines 24 (e.g., 4 or more splines 24) and any suitable number of electrodes 26 (e.g., one or more electrodes 26) per spline 24. The splines 24 and electrodes 26 are described in more detail with reference to FIG. 3A.

The resilient splines 24 are disposed circumferentially around the distal portion 20 of the puller 18 and around a longitudinal axis 25 of the catheter 10, with first ends 28 (only some labeled for the sake of simplicity) of the splines 24 being coupled with the distal end of the deflectable element (e.g., via the coupler 16) and second ends 30 (only some labeled for the sake of simplicity) of the splines 24 coupled with the distal portion of the puller 18. The ends 28 of splines 24 may be coupled to the outside of the coupler 16 or the inside of the coupler 16 using adhesive or a pressure fit, for example. The ends 30 of splines 24 may be connected together using an adhesive or via a coupler (not shown) using adhesive or a pressure fit, for example.

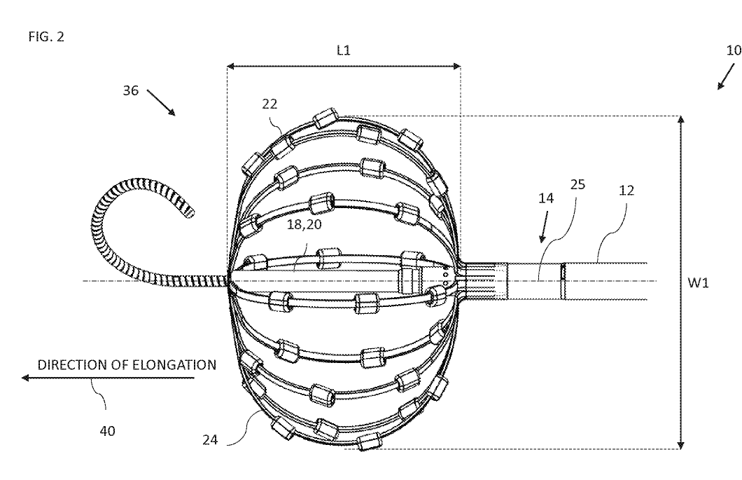

The splines 24 are configured to bow radially outward (from the distal portion 20 of the puller 18) in a relaxed form 34 of the expandable assembly 22. The relaxed form 34 of the expandable assembly 22 generally describes a pear-shaped surface described in more detail with reference to FIG. 3A. In some exemplary modes, the relaxed form 34 may have any suitable shape. The term “relaxed form”, as used in the specification and claims, is defined as a form that the expandable assembly 22 takes when no external force (external to the expandable assembly 22) is exerted on the expandable assembly 22. If an external force is exerted on the expandable assembly 22 (for example by retracting the puller 18 in the elongated deflectable element 12, or retracting the expandable assembly 22 into a sheath, or by pressing the expandable assembly 22 against tissue of a heart chamber), the splines 24 will generally deform. Once the external force is removed, the expandable assembly 22 returns to the relaxed form 34 of the expandable assembly 22 with the splines 24 bowing radially outward from the distal portion 20 of the puller 18. The splines 24 are configured to bow further radially outward when the puller 18 is retracted (arrow 32) in the elongated deflectable element 12 expanding the expandable assembly 22 from the relaxed form 34 to an expanded form 36 shown in FIG. 2. FIG. 1 also shows the distal end of a guidewire 38. The rest of the guidewire 38 is disposed in a lumen of the puller 18 and the elongated deflectable element 12. The guidewire 38 may be used to help position the catheter 10 in a body part of a living subject.

Reference is now made to FIG. 2, which is a schematic view of the catheter 10 of FIG. 1 in the expanded form 36 of the expandable assembly 22. As previously mentioned, the splines 24 are configured to bow further radially outward (from the distal portion 20 of the puller 18) around the longitudinal axis 25 when the puller 18 is retracted in the elongated deflectable element 12 expanding the expandable assembly 22 from the relaxed form 34 to the expanded form 36. The expanded form 36 of the expandable assembly 22 has a more spherical shape with a large width than the relaxed form 34 (FIG. 1) of the expandable assembly 22 and is more suited to wider circumferential ablations. Retracting the puller 18 by different amounts may result in more than two forms of the expandable assembly 22. In some exemplary modes, the splines 24 are configured to bow further radially outward when the puller 18 is retracted by around 0.5 centimeters expanding the expandable assembly 22 from the relaxed form 34 (FIG. 1) to the expanded form 36. The expanded form 36 of the expandable assembly 22 may have any suitable dimensions (e.g., length and width, maximum diameter). In some exemplary modes, an extent (e.g., length L1) of the expanded form 36 of the expandable assembly 22 measured in a direction parallel to a direction of elongation 40 of the distal end 14 is in a range of 30 to 35 millimeters. In some exemplary modes, a maximum width (e.g., width W1) of the expandable assembly 22 in the expanded form 36 is in a range of 28 to 33 millimeters.

Reference is now made to FIG. 3A, which is a more detail schematic view of the catheter 10 of FIG. 1 in the relaxed form 34. In some exemplary modes, the relaxed form 34 of the expandable assembly 22 defines a pear-shaped surface 42 around the longitudinal axis 25. The pear-shaped surface 42 comprises a convex-shaped region R1 followed by a concave-shaped region R2 in a direction progressing away from the distal end 14 of the deflectable element 12. The relative sizes of the convex-shaped region R1 and the concave-shaped region R2 may take any suitable values. In some exemplary modes, the concave-shaped region R2 is at least 10 percent of the surface area of the pear-shaped surface 42.

Reference is now made to FIG. 3B which illustrates the basket assembly 22 of FIG. 3A with certain dimensions defined. Specifically, basket 22 has a proximal end 22A that extends along a longitudinal axis 25 to a distal end 22B. Basket 22 has a plurality of spines 24 disposed angularly about the longitudinal axis 25. Each spine 24 has a plurality of electrodes disposed thereon. Of note is the teardrop shape of the basket assembly 22 in which the diameter Dmax of the basket can be determined as where diametrically outer surface of opposed spines are tangential to a axes 25A and 25 parallel to the longitudinal axis 25, indicated here at T1 and T2. A line connecting T1 and T2 perpendicular to axis 25 at center C defines the maximum diameter of the basket assembly 22. Because of the teardrop configuration, it can be seen that a first distance LL1 from the distal end 22B of the basket 22 to the center C of Dmax is greater than the second distance LL2 from the proximal end 22A to the center of Dmax. First length LL1 may have twice the magnitude of second length LL2 in the relaxed configuration of basket 22.

When the puller 18 is pulled proximally (towards the operator) the basket assembly 22 moves from a teardrop shape to a spheroidal shape shown in FIG. 3C. In FIG. 3C, it can be seen that the first length LL1 from the distal end 22B to the center C of the maximum diameter Dmax is approximately equal to the second length LL2 from the center C to the proximal end 22A.

The splines 24 may be formed from any suitable material or materials. In some exemplary modes, each spline 24 includes a spring alloy elongated element 44 extending from the end 28 connected to the coupler 16 to the end 30 connected to the other splines 24. In some exemplary modes, the spring alloy elongated element 44 includes a nickel titanium (e.g., Nitinol®) elongated element, or a spring tempered stainless steel elongated element, or a beryllium copper (BeCu)) elongated element. The splines may have any suitable dimensions which provide the expandable assembly 22 with both suitable resilience and flexibility. In one example, the splines are about 0.004 inches thick and about 0.022 inches wide. The splines may have any suitable cross-section, e.g., square, square with rounded edges, or curved. Each spline 24 may include a polymer tube 48 (e.g., formed from polyether block amide (such as PEBAX®, polyurethane, styrene-ethylene-butylene-styrene thermoplastic elastomer (SEBS), and/or polyethylene terephthalate (PET)) at least partially covering the spring alloy (e.g., nickel titanium) elongated element 44. A cross-section of one of the splines 24 is shown in inset 46. An inset 50 shows a cross-sectional view of the distal end of the expandable assembly 22. The inset 50 shows the spring alloy elongated elements 44 of the splines 24 being connected to the puller 18. The inset 50 also shows the guidewire 38 in the lumen of the puller 18.

In some exemplary modes, the electrode(s) 26 are mounted over the polymer tube 48. A respective wire (not shown) is connected to the inner surface of each respective electrode 26. The respective wire is fed through a respective hole (not shown) in the polymer tube 48 and through the lumen of the polymer tube 48 to the coupler 16 or the elongated deflectable element 12. The wires from the different electrodes 26 may then run through the elongated deflectable element 12 to a proximal end of the catheter 10. The electrodes 26 may be formed from any suitable material, for example, gold or stainless steel, and have any suitable shaped. In some exemplary modes, the electrode 26 are biased to have more bulk facing outside the expandable assembly 22.

The relaxed form 34 of the expandable assembly 22 may have any suitable dimensions (e.g., length and width, maximum diameter). In some exemplary modes, an extent (e.g., length L2) of the relaxed form 34 of the expandable assembly 22 measured in a direction parallel to the direction of elongation 40 of the distal end 14 is in a range of 35 to 45 millimeters. In some exemplary modes, a maximum width (e.g., W2) of the expandable assembly 22 in the relaxed form 34 is in a range of 25 to 30 millimeters.

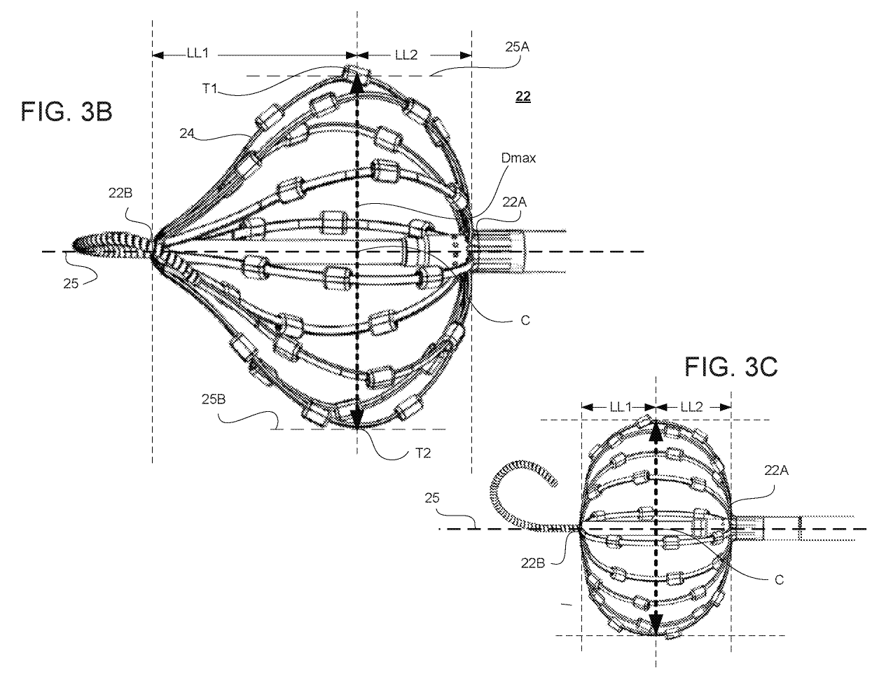

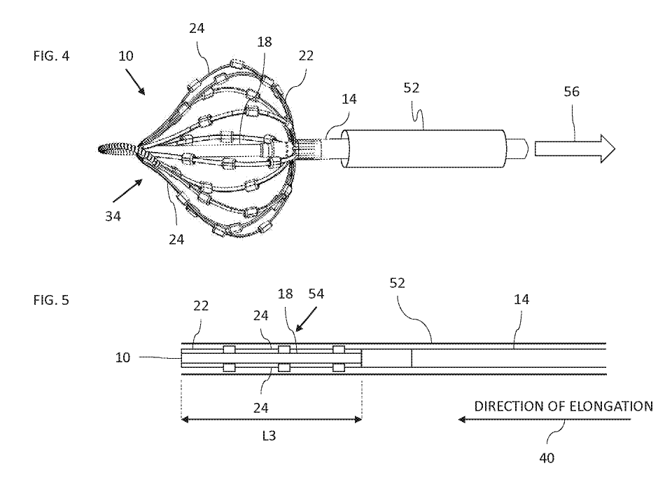

Reference is now made to FIGS. 4 and 5. FIG. 4 is a schematic view of the catheter 10 of FIG. 1 being retracted into a sheath 52. FIG. 5 is a schematic cross-sectional view of the catheter 10 of FIG. 1 retracted into the sheath 52 of FIG. 4. The splines 24 are configured to flatten and transform the relaxed form 34 of the expandable assembly 22 to a collapsed form 54 of the expandable assembly 22 when the expandable assembly 22 is pulled (arrow 56) into the sheath 52, which applies a radial compressive force on the splines 24. In some exemplary modes, an extent (e.g., length L3) of the collapsed form 54 measured in a direction parallel to the direction of elongation 40 of the distal end 14 is in a range of 35 to 45 millimeters.

In some exemplary modes, the puller 18 is stretchable and is configured to stretch as the splines 24 are flattened. In some disclosed modes, the puller 18 includes a stretchable woven tube. The stretchable woven tube may be formed from a braided polymer tube, including for example, a substrate of PEBAX with an LCP filament or yarn used for braiding and then PEBAX film cast over the braiding. Suitable polymer tubes are commercially available from Putnam Plastics Corporation Dayville, CT 06241, United States, and MicroLumen high performance medical tubing, Oldsmar, FL 34677 USA.

In some disclosed modes, the puller 18 may be a stretchable wire. In some exemplary modes, the puller 18 is stretchable by about 1 percent. For example, if the catheter 10 is about 1 meter long and the length of the assembly increases by 1 centimeter between the relaxed form 34 and the compressed form 54, the puller 18 needs to stretch by about 1%. In some exemplary modes, the puller is stretchable by at least 0.5 percent.

As used herein, the terms "about" or "approximately" for any numerical values or ranges indicate a suitable dimensional tolerance that allows the part or collection of components to function for its intended purpose as described herein. More specifically, "about" or "approximately" may refer to the range of values ±20% of the recited value, e.g. "about 90%" may refer to the range of values from 72% to 108%.

### EXAMPLES

Example 1: A catheter apparatus (10), comprising: an elongated deflectable element (12) including a distal end (14); a flexible puller (18) including a distal portion (20), and configured to be retracted through the deflectable element; and an expandable assembly (22) comprising a plurality of resilient splines (24), each resilient spline including at least one electrode (26) disposed thereon, the resilient splines being disposed circumferentially around the distal portion of the puller, with first ends (28) of the splines being coupled with the distal end of the deflectable element and second ends (30) of the splines coupled with the distal portion of the puller, the splines being configured to: bow radially outward in a relaxed form of the expandable assembly: and bow further radially outward when the puller is retracted expanding the expandable assembly from the relaxed form (34) to an expanded form (36).

Example 2: The apparatus according to example ‎1, wherein an extent (L1) of the expanded form measured in a direction parallel to a direction of elongation (40) of the distal end is in a range of 30 to 35 millimeters.

Example 3: The apparatus according to example ‎1 or 2, wherein an extent (L2) of the relaxed form measured in the direction parallel to the direction of elongation of the distal end is in a range of 35 to 45 millimeters.

Example 4: The apparatus according to any of examples ‎1-3, wherein a maximum width (W1) of the assembly in the expanded form is in a range of 28 to 33 millimeters.

Example 5: The apparatus according to any of examples ‎1-4, wherein a maximum width (W2) of the assembly in the relaxed form is in a range of 25 to 30 millimeters.

Example 6: The apparatus according to any of examples ‎1-5, wherein the splines are configured to flatten and transform the relaxed form to a collapsed form (54) of the expandable assembly when the expandable assembly is pulled into a sheath (52), which applies a radial compressive force on the splines.

Example 7: The apparatus according to example ‎6, wherein an extent (L3) of the collapsed form measured in a direction parallel to a direction of elongation of the distal end is in a range of 35 to 45 millimeters.

Example 8: The apparatus according to example ‎6 or 7, wherein the puller is stretchable and is configured to stretch as the splines are flattened.

Example 9: The apparatus according to any of examples 1-‎8, wherein the puller is stretchable by at least 0.5 percent.

Example 10: The apparatus according to any of examples 1-‎8, wherein the puller is stretchable by about 1 percent.

Example 11: The apparatus according to any of examples 1-‎8, wherein the puller includes a stretchable woven tube.

Example 12: The apparatus according to any of examples ‎1-11, wherein the relaxed form of the expandable assembly defines a pear shape surface (42).

Example 13: The apparatus according to example ‎12, wherein the pear-shaped surface comprises a convex-shaped region (R1) followed by a concave-shaped region (R2) in a direction progressing away from the distal end of the deflectable element.

Example 14: The apparatus according to example ‎13, wherein the concave-shaped region is at least 10 percent of the surface area of the pear-shaped surface.

Example 15: The apparatus according to any of examples ‎1-14, wherein each resilient spline includes a nickel titanium elongated element (44).

Example 16: The apparatus according to example ‎15, wherein each resilient spline includes a polymer tube (48) at least partially covering the nickel titanium elongated element.

Example 17: The apparatus according to any of examples ‎1-16, wherein the splines are configured to bow further radially outward when the puller is retracted by around 0.5 centimeters expanding the expandable assembly from the relaxed form to the expanded form.

Example 18: A basket catheter that includes a plurality of spines disposed radially around a longitudinal axis to define a basket assembly extending from a proximal end to a distal end along the longitudinal axis, the basket assembly defining a teardrop shape having a maximum diameter as measured orthogonally to a center disposed on the longitudinal axis, the basket assembly having a first length from the distal end to the center of the maximum diameter of the basket assembly and a second length as measured from the center of the maximum diameter along the longitudinal axis to the proximal end of the basket assembly, the first length being greater than the second length.

Example 19: The basket catheter of example 18 in which the first length is approximately twice the second length.

Example 20: The basket catheter of example 19 in which a plurality of electrodes is disposed on each spine.

Various features of the disclosure which are, for clarity, described in the contexts of separate examples may also be provided in combination in a single example. Conversely, various features of the disclosure which are, for brevity, described in the context of a single example may also be provided separately or in any suitable sub-combination.

The examples described above are cited by way of example, and the present disclosure is not limited by what has been particularly shown and described hereinabove. Rather the scope of the disclosure includes both combinations and sub-combinations of the various features described hereinabove, as well as variations and modifications thereof which would occur to persons skilled in the art upon reading the foregoing description and which are not disclosed in the prior art.

What is claimed is:

1. A catheter comprising:
an elongated deflectable element extending along a longitudinal axis and including a distal end; and
an expandable assembly comprising a plurality of splines disposed circumferentially around the longitudinal axis, each spline including at least one electrode disposed thereon, with first ends of the plurality of splines being coupled with to the distal end of the elongated deflectable element and second ends of the plurality of splines coupled together, the plurality of splines being configured to:
flatten into a collapsed form;
bow radially outward in a relaxed form of the expandable assembly defining a pear-shaped surface; and
bow further radially outward in an expanded form defining a spheroidal-shaped surface extending from a distal end to a proximal end of the expandable assembly.

2. The catheter of claim 1, further comprising a puller configured to be retracted through the elongated deflectable element and comprising a distal portion coupled to the second ends of the plurality of splines such that the splines are disposed circumferentially around the distal portion of the puller, wherein the plurality of splines are configured to:
bow radially outward in the relaxed form when no external forces are exerted on the expandable assembly;
bow further radially outward in the expanded form when the puller is retracted proximally from the relaxed form; and
flatten and transform the expandable assembly from the relaxed form to the collapsed form when the expandable assembly is pulled into a sheath.

3. The catheter of claim 2, wherein the puller is stretchable and configured to stretch as the plurality of splines are flattened.

4. The catheter according to claim 3, wherein the puller is stretchable by at least 0.5 percent.

5. The catheter of claim 3, wherein the puller comprises a braided polymer tube.

6. The catheter of claim 2, wherein the puller comprises a lumen configured to receive a guidewire.

7. The catheter according to claim 2, wherein the splines are configured to bow further radially outward when the puller is retracted by around 0.5 centimeters expanding the expandable assembly from the relaxed form to the expanded form.

8. The catheter of claim 1, the pear-shaped surface comprising a convex-shaped region followed by a concave-shaped region in a direction progressing away from the distal end of the elongated deflectable element.

9. The catheter of claim 8, wherein the concave-shaped region is at least 10 percent of a surface area of the pear-shaped surface.

10. The catheter of claim 8, further comprising a puller configured to be retracted through the elongated deflectable element and comprising a distal portion coupled to the second ends of the plurality of splines such that the splines are disposed circumferentially around the distal portion of the puller.

11. The catheter of claim 10, wherein each spline comprises a spring alloy elongated element, such that the plurality of splines are configured to:
bow radially outward in the relaxed form when no external forces are exerted on the expandable assembly;
bow further radially outward in the expanded form when the puller is retracted proximally from the relaxed form; and
flatten and transform the expandable assembly from the relaxed form to the collapsed form when the expandable assembly is pulled into a sheath.

12. The catheter of claim 11, wherein each spline includes a polymer tube at least partially covering the spring alloy elongated element, wherein the at least one electrode is mounted on the polymer tube.

13. The catheter according to claim 1, wherein, in the expanded form, a distance from the proximal end to the distal end of the expandable assembly measured in a direction parallel to a direction of elongation is 30 millimeters to 35 millimeters.

14. The catheter according to claim 1, wherein, in the relaxed form, a distance from the proximal end to the distal end of the expandable assembly measured in a direction parallel to a direction of elongation is 35 millimeters to 45 millimeters.

15. The catheter according to claim 1, wherein a maximum width of the expandable assembly in the expanded form is in a range of 28 millimeters to 33 millimeters.

16. The catheter according to claim 1, wherein a maximum width of the expandable assembly in the relaxed form is in a range of 25 millimeters to 30 millimeters.

17. The catheter according to claim 1, wherein, in the collapsed form a distance from the proximal end to the distal end of the expandable assembly measured in a direction parallel to a direction of elongation is 35 to 45 millimeters.

18. The catheter according to claim 1, wherein a first distance from the distal end of the expandable assembly to a maximum diameter of the expandable assembly in the relaxed form is greater than a second distance from the proximal end of the expandable assembly to the maximum diameter in the relaxed form, and wherein the first distance is approximately equal to the second distance in the expanded form.

19. A basket catheter comprising:
a plurality of spines disposed radially around a longitudinal axis to define a basket assembly extending from a proximal end to a distal end along the longitudinal axis, the basket assembly comprising a maximum diameter as measured orthogonally to the longitudinal axis, a first length measured along the longitudinal axis from the proximal end to the maximum diameter, and a second length measured along the longitudinal axis from the maximum diameter to the distal end, the basket catheter comprising:
a first configuration defining a teardrop shape with the first length being greater than the second length; and
a second configuration defining a spheroidal-shaped surface extending from the proximal end to the distal end with the first length being approximately equal to the second length.

20. The basket catheter of claim 19, further comprising a puller coupled to the distal end of the basket catheter, wherein the basket catheter is transitioned from the first configuration to the second configuration as the puller is moved toward the proximal end.
